(19) United States
(12) Patent Application Publication
(10) Pub. No.: US 2004/0137200 A1
(43) Pub. Date: Jul. 15, 2004

(54) Nonwoven wipe with resilient wet thickness

(75) Inventors: Rajeev Chhabra, Mason, OH (US); Ronald Thomas Gorley, Cincinnati, OH (US); Jeffery Len Osborne, Harrions, OH (US); Jonathan Paul Brennan, Cincinnati, OH (US); Ali Abdelaziz Alwattari, Pointe Claire (CA)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/712,239

(22) Filed: Nov. 13, 2003

Related U.S. Application Data
(60) Provisional application No. 60/425,963, filed on Nov. 13, 2002.

(51) Int. Cl. B32B 1/00; D06N 7/04; B32B 3/28; B32B 3/30; B29C 67/20; B32B 27/14; B29C 33/40; B27N 3/08; B28B 19/00; B29C 51/00; B29C 53/00; A61M 35/00; D04H 1/00; D04H 3/00; D04H 5/00; D04H 13/00

(52) U.S. Cl. 428/167; 428/141; 428/152; 428/198; 604/289; 264/175; 264/239; 264/222

(57) ABSTRACT

Nonwoven substrates suitable for use as wet wipes are disclosed. The nonwoven substrates comprise at least one first region and at least one second region. The second region comprises reinforced protruding elements. In a preferred embodiment, the second region of the nonwoven substrate is reinforced by means of thermal bonding during the creation of the protruding elements of the second region. A liquid can be added to the nonwoven substrate prior to packaging or prior to use to make wet wipes. The reinforced second regions of the nonwoven substrates of the present invention make it possible to retain thickness of the wipe when wet without increasing the dry basis.

### CROSS REFERENCE TO RELATED APPLICATION

This application claims the benefit of U.S. Provisional Application No. 60/425,963, filed Nov. 13, 2002

### FIELD OF INVENTION

The present invention is related to nonwoven substrates, and more particularly to wet wipes suitable for both personal hygiene and surface applications.

### BACKGROUND

Wet wipes are well known in the art. Wet wipes include a substrate, such as a nonwoven web, and a liquid. Sometimes, the liquid is applied by the manufacturer and sold to the consumer as a pre-moistened wet wipe. At other times, the wipe is sold to the consumer dry and the consumer adds their own liquid to create a wet wipe. In the case where the wipe is sold to the consumer dry and the consumer adds their own liquid, the nonwoven web may include active ingredients that combine with the liquid that the consumer adds. The liquid that the consumer adds can be water or another liquid such as a lotion.

Examples of pre-moistened wet wipes for hygiene use include Pampers® Baby Wipes, Charmin® Fresh Cloths, Olay® Wipes, and Old Spice™ Refreshment Towels, sold by The Procter & Gamble Company. Examples of pre-moistened wet wipes for surface use include Mr. Clean® and Mr. Propre® Cleaning Wipes, sold by The Procter & Gamble Company. An example of a wet wipe, where the consumer adds their own liquid is Olay® Daily Facials™, sold by The Procter & Gamble Company.

Various nonwoven substrates are used to make wet wipes. A variety of forming technologies are used to make these nonwoven substrates, including carding, airlaid, spunbond, meltblown, coform, and wetlaid. Various consolidation technologies are also used to make the nonwoven substrates, including hydroentanglement, thermal calender bonding, through air thermal bonding, chemical bonding, and needlepunching. Fibrous materials are used in the making of these nonwoven substrates, including thermoplastic fibers, natural fibers, and cellulosic fibers. Thermoplastic fibers include polyolefins (e.g., polyethylene and polypropylene), polyesters, polyamides, polyimides, polyacrylates, polyacrylonitrile, polylactic acid, polyhydroxyalkanoate, polyvinyl alcohol, polystyrene, polyaramids, polysaccharides and blends and co-polymers thereof. Natural fibers include cotton, wool, silk, jute, linen, ramie, hemp, flax, camel hair, kenaf, and mixtures thereof. Cellulosic fibers include wood pulp, rayon, lyocell, cellulose acetate, cellulose esters and mixtures thereof.

There are several known methods to increase the thickness and/or texture of a dry wipe. However, a nonwoven substrate typically decreases in thickness when wetted with liquid as it is transformed into a wet wipe. Thickness in a wet wipe is often a desirable attribute so methods to increase the wet wipe thickness are desired. One method to increase thickness is to add basis weight to the nonwoven substrate. Adding basis weight, or more material to the nonwoven substrate, increases the dry thickness of the nonwoven substrate and the wet thickness of the wet wipe. One disadvantage of adding basis weight is incremental cost. Another method to increase thickness by increasing basis weight is disclosed in WO 02/076723 A1 by Walton, et. al. The “dry creping” process disclosed in WO 02/076723 A1 shortens the web effectively increasing the overall basis weight of the nonwoven web. It would be more cost effective to have a thickness increasing process that does not result in an increase in basis weight.

Another problem that exacerbates the difficulty in retaining wet thickness and texture is that wet wipes, and especially pre-moistened wet wipes, are subject to hydrodynamic and compression forces that tend to reduce the wet thickness and texture.

It is an object of this invention to overcome the typical problems of retaining wet thickness and texture in a wet wipe. Specifically, it is an object of the present invention to provide a nonwoven substrate that retains the thickness when wet and preferably, when subjected to external forces such as hydrodynamic and compression, without increasing the dry basis weight.

### SUMMARY OF INVENTION

Nonwoven substrates suitable for use as wet wipes are disclosed. The nonwoven substrates comprise at least one first region and at least one second region. The second region comprises reinforced protruding elements. In a preferred embodiment, the second region of the nonwoven substrate is reinforced by means of thermal bonding during the creation of the protruding elements of the second region. A liquid can be added to the nonwoven substrate prior to packaging or prior to use to make wet wipes. The reinforced second regions of the nonwoven substrates of the present invention make it possible to retain thickness of the wipe when wet without increasing the dry basis weight.

The present invention also relates to wet wipes comprising a nonwoven substrate which is subject to a texturing process which does not increase the basis weight and a liquid. Preferably, the thickness of the wet wipe of the present invention is at least about 30% greater than the thickness of a wet (non-textured) nonwoven substrate and of a wet textured nonwoven substrate that is produced by traditional texturing methods that do not create reinforced second regions. It is also preferred that the thickness of the wet wipe of the present invention after being subject to external forces is at least about 30% greater than the thickness of the wet non-textured nonwoven substrate after being subject to external forces and of a wet textured nonwoven substrate that is produced with traditional texturing methods that do not create reinforced second regions after being subject to external forces.

The present invention also relates to a process for providing texture and increasing thickness to the above nonwoven substrate comprising feeding the substrate through a pair of corresponding rolls, wherein at least one of the pair of rolls comprises a plurality of toothed and grooved regions about the circumference of the rolls. The grooved roll regions form the first regions of the substrate and the toothed roll regions form the second regions of the substrate. In a preferred embodiment, the rolls are heated thereby enabling reinforcing of the second regions of the nonwoven substrate of the present invention.

### BRIEF DESCRIPTION OF THE DRAWINGS

While the specification concludes with claims particularly pointing out and distinctly claiming the present invention, it is believed that the present invention will be better understood from the following description taken in conjunction with the accompanying drawings in which like reference numerals identify like elements.

FIG. 1 is a plan view illustration of a preferred embodiment of the substrate of the present invention showing the diamond shaped second regions.

FIG. 2 is a scanning electron micrograph of a preferred embodiment with a typical thermal lock formed during the creation of the first and second regions in the starting substrate of the preferred embodiment.

FIG. 3 is a scanning electron micrograph of a preferred embodiment with typical thermal locks unbroken during the creation of the first and second regions in the starting substrate of the preferred embodiment.

FIG. 3a is a scanning electron micrograph showing cross-sectional view of ridges and furrows of the reinforced protruding elements of second regions of the preferred embodiment substrate shown in FIG. 3.

FIG. 4 is a simplified perspective view of a preferred apparatus used to form substrates of the present invention with a portion of the apparatus being tilted to expose the teeth.

FIG. 5 is a simplified side elevation view of a static press used to form the substrate of the present invention.

FIG. 6 is a simplified side elevation view of a continuous, dynamic press used to form the substrates of the present invention.

FIG. 7 is a simplified illustration of another apparatus used to form the substrates of the present invention.

FIG. 7a is a blown up illustration of the boxed area in FIG. 7, showing the distance of depth of engagement (DOE) of two corresponding rolls.

FIG. 8 is another simplified illustration of another apparatus used to form the substrates of the present invention.

FIG. 9 is a plan view illustration of a preferred embodiment of the substrates of the present invention showing second regions in a diamond shape comprising reinforced protruding elements.

FIG. 10 is a plan view illustration of another preferred embodiments of the substrates of the present invention showing second regions in a diamond shape comprising reinforced protruding elements.

### DETAILED DESCRIPTION OF THE INVENTION

Substrate

As used herein, the term “substrate” means a single web or a laminate of two or more webs. The term web means a fibrous web. A starting or initial substrate means the substrate prior to texturizing or mechanical manipulation thereof.

Wet Wipe

Wet wipes means a substrate, such as a nonwoven web, that is utilized when wet. The wipe is made wet by the addition of a liquid. A liquid can be applied by the manufacturer prior to packaging and sold to the consumer as a pre-moistened wet wipe. A wet wipe can also be sold to the consumer dry and the consumer adds their own liquid to the wipe. In the case where the wipe is sold to the consumer dry and the consumer adds their own liquid, the nonwoven web may include active ingredients that combine with the liquid that the consumer adds. The liquid that the manufacturer or consumer adds can be water or another liquid such as a lotion.

The First and Second Regions

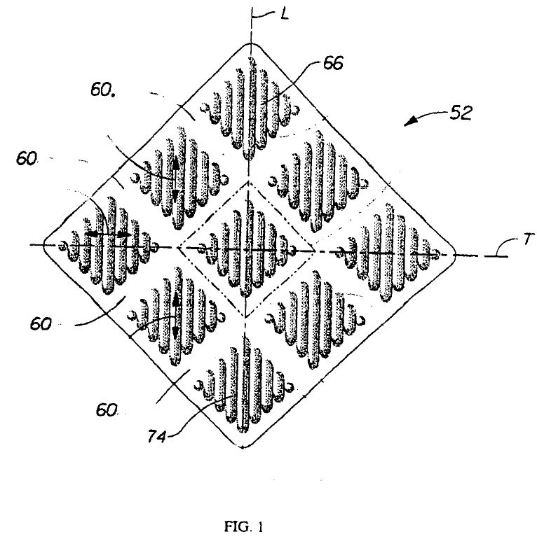

The substrates of the present invention comprise at least a first region and at least a second region. Preferably said substrates comprise a plurality of first and second regions. FIG. 1 shows substrate 52, an embodiment of the present invention, with the first regions 60 and the second regions 66. Said second regions are capable of greater geometric deformation than said first regions. As used herein the term “geometric deformation” refers to deformations of the substrate, which are generally discernible to the normal naked eye when the substrate or articles embodying the substrate are subjected to an applied force. This is in contrast to “molecular-level deformation” which refers to deformation, which occurs on a molecular level and is not discernible to the normal naked eye. That is, even though one may be able to discern the effect of molecular-level deformation, e.g., elongation of the substrate, one is not able to discern the deformation, which allows or causes it to happen.

The first regions are preferably and most typically visually distinct from the second regions as shown in FIG. 1. As used herein, the term “visually distinct” refers to features of the substrate, which are readily discernible to the normal naked eye when the substrate or objects embodying the substrate are subjected to normal use. Referring to FIG. 1, the first regions 60, when compared to the second regions 66, are substantially planar and unformed. The function of such areas is to provide integrity and strength to the substrate, especially during use. In comparison to the second regions, the first regions are less geometrically deformable. Hence, while the first regions may also undergo such geometric deformation, it is less than what is discernible with respect to the second regions of the substrate. Thus, the main role of the first regions of the substrate of the present invention is to limit the degree of geometric deformation of the substrate per se.

The second regions by contrast comprise protruding elements 74, which are formed during the texturizing process described below. As used herein, the term “protruding element” refers to an area of formation of ridges and/or furrows on the surface of the substrate. The protruding elements may appear visually like a region of corrugation. The formation may be above or below the plane of the substrate and may be convex and/or concave. The protruding elements may consist of only slight formation of the substrate, producing a mildly undulating surface. Preferably, the protruding elements are more pronounced however and can be described as rib-like elements. Rib-like elements comprise a major axis and a minor axis defining an elongated cubical, ellipsoidal or other similar rib-like shape. The major axis and the minor axis of the protruding rib-like elements may each be linear, curvilinear or a combination of linear and curvilinear. Each second region of the substrate preferably comprises a plurality of protruding elements. More preferably the protruding elements in each second region are contiguous with no unformed or first regions between them.

The protruding elements of the second region permit greater geometric deformation. Types of geometric deformation include, but are not limited to bending, folding, unfolding, and rotating. Since these protruding elements are capable of greater geometric deformation than the first regions, it is the object of the present invention to “lock” the fibers of the protruding elements of the second regions to better resist the geometric deformation. As referred herein, the term “locking” means physically constraining fibers in the second regions, leading to preservation of the protruding elements of the second regions of the substrate after being subjected to the “external forces”. In the absence of locking, when an “external force” is applied to the second region of the substrate, the protruding areas are compressed, stretched, extended or deformed, becoming more planar, to the point of being substantially planar like the first regions after the “external force” is removed. In contrast, in the substrate of the present invention, said protruding elements are “reinforced” and substantially resilient, meaning that the substrate substantially reforms its original shape and caliper after the applied external force to the substrate is removed. As used herein, the term “reinforce” means strengthening of protruding elements by locking of fibers in the second regions of the substrate and thereby, providing increased resistance to geometrical deformation. The amount of thickness recovery (caliper rebound) exhibited by the substrate is a measure of the substrate's structural permanence after the applied external force is removed. A method to measure the wet structural permanence of a substrate of the present invention is described later in the Test Methods section. Types of “external forces” include, but are not limited to, hydrodynamic, compression, tension, shear, and mixtures thereof. Types of reinforcement means include, but are not limited to thermal bonding, chemical bonding, ionic bonding, adhesive bonding, and combinations thereof. The reinforcement or lock may be formed during the texturing process of forming the first and second regions. In a preferred embodiment, reinforcing or locking of fibers occurs via thermal bonding of fibers during the creation of the first and second regions in the starting substrate.

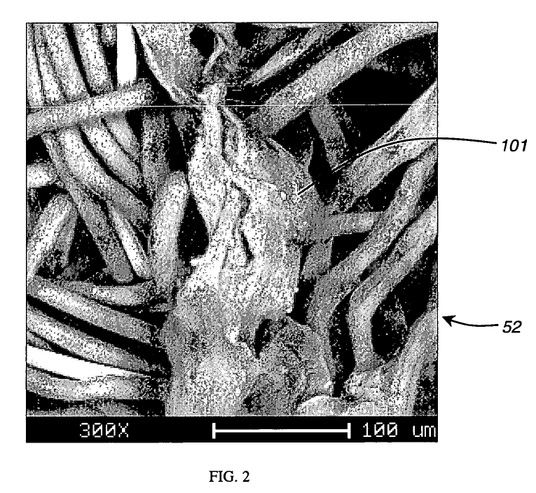

FIG. 2 shows a scanning electron micrograph of a fibrous substrate 52 with a typical thermal lock 101 formed during the creation of the first and second regions in the starting substrate of a preferred embodiment. Typically, this is a prebonded web. When the starting substrates having the locks, exemplified by but not limited to thermal or adhesive bonds, is mechanically manipulated to form the first and second regions, a substantial number of locks are “unbroken” by the texturing process, thereby reinforcing the second regions. As described herein, the term “unbroken” means being substantially intact physically and/or chemically.

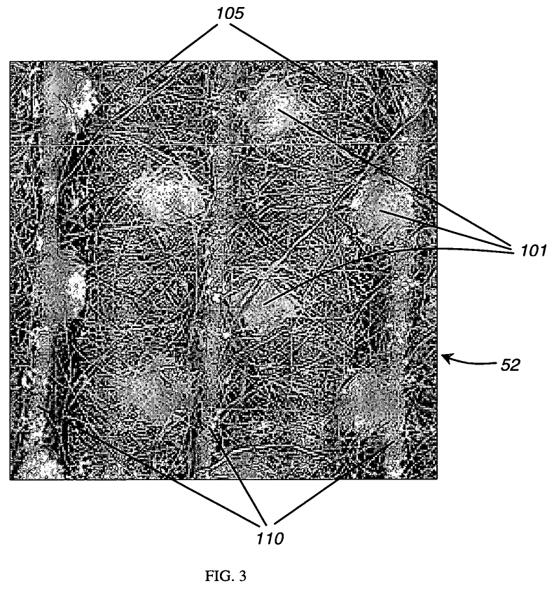

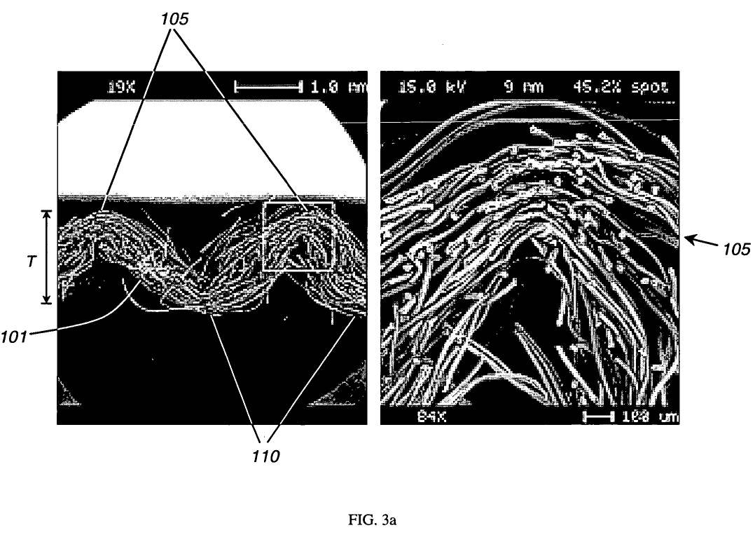

FIG. 3 shows a scanning electron micrograph of a substrate 52 with typical thermal locks 101 unbroken during the creation of the first and second regions in the starting substrate. FIG. 3 also shows ridges 105 and furrows 110 of the reinforced protruding elements of the second regions in a preferred embodiment substrate of the present invention. FIG. 3a shows the cross-sectional view of ridges 105 and furrows 110, or rib-like elements, of the reinforced protruding elements of the second regions of the preferred embodiment substrate shown in FIG. 3. Also shown in FIG. 3a is a typical thermal lock 101 unbroken during the creation of the first and second regions in the starting substrate.

Because the rib-like elements are protruding from the plane of the substrate, they effectively increase the thickness of the substrate as compared to the non-textured starting substrate. Furthermore, the method of forming the protruding elements of the second region (as explained later) is such that the dry basis weight of the substrate is substantially unchanged. The method to measure the basis weight of the substrates is described later the Test Methods section. The locking of fibers in the second regions preserves the protruding elements even when the substrate is wetted with a liquid to form a wet wipe, and thus the wet thickness of the substrate of the present invention is greater than that of the starting substrate. Depending on the amount of extension of the protruding elements from the surface plane of the substrate and the strength of locking, the wet thickness of the wet wipe of the present invention ranges from about 110% to about 300% compared to the same wet wipe substrate without the second regions (i.e., with only the first regions). The wet thickness is measured by a method described later in the Test Methods section.

The first and second regions may be of any suitable shape and arranged in any desirable pattern. Examples of shapes may include strips, waves, or blocks of first and second regions intermittently spaced or islands of second regions in first regions or vice versa. In one preferred embodiment strips of the first regions are intermittently spaced between strips of second regions. In another preferred embodiment a portion of the first regions extend in a first direction while the remainder of the first regions extends in a second direction such that the first regions extending in different directions intersect one another at intervals. The second direction is preferably substantially perpendicular to the first direction. In this embodiment the first regions form a boundary completely surrounding the second regions, such that the overall pattern of first and second regions formed resembles a plurality of diamonds (FIGS. 1, 9, and 10). The percentage surface area coverage of the substrate of first and second regions may vary according to the intended use and pattern desired.

The first and second regions provide a texture to the wipe that is retained when the wipe is wet. This added texture provides depth, thickness, loft, pockets, softness and/or abrasivity to a wipe used when wet. The texture that remains when the wet wipe is used provides enhanced cleaning. The greater the amount of surface area of the substrate that has texture, the greater the cleaning benefit. Additionally, the texture provided to the wet wipe also provides increased consumer perception of improved cleaning.

Method of Making the Substrates

The substrates of the present invention comprise first and second regions. As discussed above the first regions are substantially unformed or planar, whereas the second regions are formed, comprising protruding elements. The first and second regions of the substrate are formed from a starting substrate that is substantially planar. Said starting substrate is fed through machinery which forms the protruding elements of the substrate in predefined areas resulting in the second regions of the substrate. Said machinery or attachments to said machinery can also reinforce fibers in the second regions of the substrate by the addition of various forms of “energy” to the substrate. Forms of said energy include but are not limited to heat, ultrasound, electromagnetic, hydrodynamic, and aerodynamic energy. Types of electromagnetic energy forms include but are not limited to ultraviolet light, infrared light, radio-frequency waves, microwaves, and electron beam. Without being bound by theory, it is believed that said addition of energy activates at least one of the components of the starting substrate and thus, enabling locking of fibers in the second regions of the substrate of present invention. Types of activation of components of starting substrate include but are not limited to melting, cross-linking, polymerization, chemical bonding, and ionic bonding. A preferred embodiment of the present invention uses machinery utilizing heat energy to lock fibers in the second regions. It will be readily apparent to those skilled in the art that the machinery of the preferred embodiment can be modified to utilize other forms of energy as noted above. These modifications are expressly intended to be within the scope of the present invention.

The processes below are described with respect to texturizing a starting substrate. Said substrate once texturized may be used as a wet wipe as is or may be a component of a more complex laminated wet wipe. In the present description, by the term textured substrate (e.g. the substrate is textured) it is meant that the starting substrate has been fed through the machinery described and the protruding elements of the second regions of the substrate have been formed.

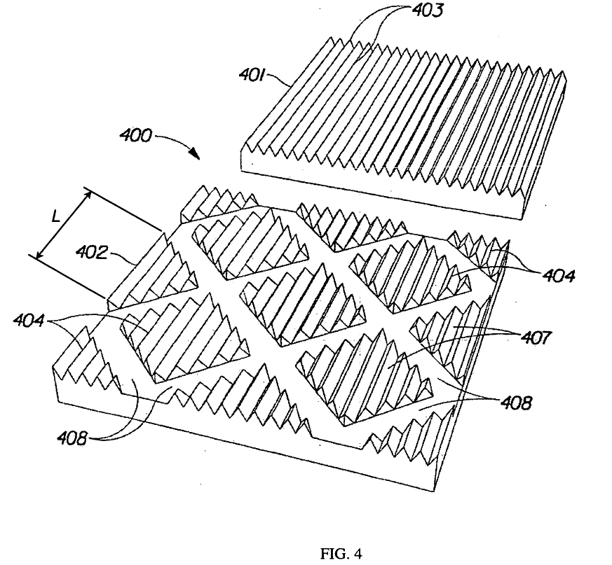

FIG. 4 shows an apparatus 400 used to form the substrate 52 shown in FIG. 1. Apparatus 400 includes intermeshing plates 401, 402. Plates 401, 402 include a plurality of intermeshing teeth 403, 404, respectively. Plates 401, 402 are brought together under pressure to form the first and second regions in the starting substrate. Plate 402 includes toothed regions 407 and grooved regions 408 both of which extend substantially parallel to the longitudinal axis of the plate 401. Within toothed regions 407 of plate 402 there are a plurality of teeth 404. Plate 401 includes teeth 403, which mesh with teeth 404 of plate 402. When a substrate is formed between plates 401 and 402, the portions of the starting substrate that are positioned within grooved regions 408 of plate 402 and teeth 403 on plate 401 remain undeformed. These regions correspond with the first regions 60 of the substrate 52 shown in FIG. 1. The portions of the starting substrate positioned between toothed regions 407 of plate 402, (which comprise teeth 404), and teeth 403 of plate 401 are incrementally formed creating the second regions and/or the protruding elements 74 in the second regions 66 of the substrate 52 shown in FIG. 1. Without being bound by theory, it is believed that the second regions are formed by straining of the starting substrate positioned between teeth 403 of plate 401, meaning that the mass, which is contained in the sections that form the second regions, extends beyond the plane of the substrate. Though by the formation of the protruding elements, the surface area of the substrate increases in the second regions, but the overall length and width of the substrate are substantially unchanged. Therefore, the dry mass per unit area (basis weight) of the whole substrate remains substantially unchanged. In a preferred embodiment, to lock fibers in the second regions of the formed substrate, the plates 401 and 402 are heated to about the melting temperature of one of the component fibers of the starting substrate. The amount of heat added is dependent upon the composition of the web. In another preferred embodiment, at least one of the plates 401 and 402 is heated to about the melting temperature of one of the component fibers of the starting substrate. Without being bound by theory, it is believed that in the preferred embodiment, heat provides the energy to melt the fibers locally in the starting substrate for creating the locks as illustrated by the scanning electron micrograph in FIG. 2.

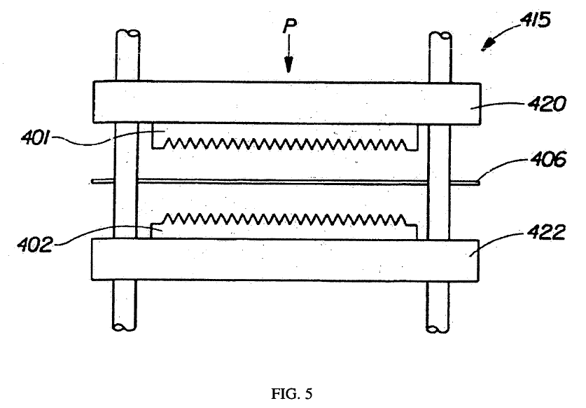

The method of texturizing can be accomplished in a static mode, where one discrete portion of a substrate is formed at a time. An example of such a method is shown in FIG. 5. A static press indicated generally as 415 includes an axially moveable plate or member 420 and a stationary plate 422. Plates 401 and 402 are attached to members 420 and 422, respectively. While plates 401 and 402 are separated, the starting substrate 406 is introduced between the plates 401 and 402. The plates are then brought together under a pressure indicated generally as “P”. Without being bound by theory, it is believed that the applied pressure “P” is dependent on the compressive and tensile strength of the starting substrate and the pattern of the toothed regions relative to the grooved regions of the plate 402. The upper plate 401 is then lifted axially away from the plate 402 allowing the textured substrate to be removed from between the plates 401 and 402. In a preferred embodiment, to lock fibers in the second regions of the textured substrate, the plate 401 and/or plate 402 are heated to about the melting temperature of one of the components of the starting substrate. Alternatively, the substrate can be heated prior to the texturizing process.

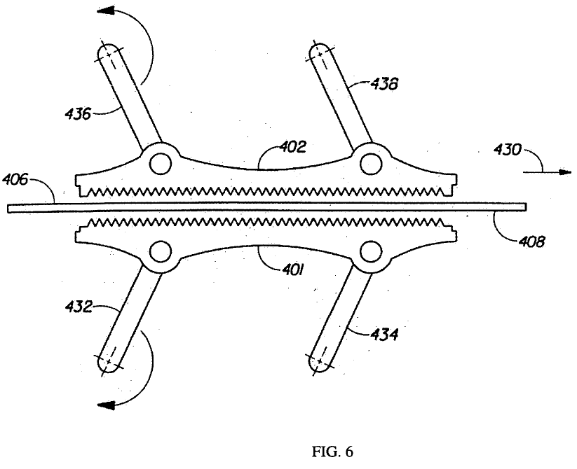

Alternatively, the method of texturizing can be accomplished using a continuous, dynamic press for intermittently contacting the moving starting substrate and forming the starting substrate into the textured substrate of the present invention. As shown in FIG. 6, the starting substrate 406 is fed between plates 401 and 402 in a direction generally indicated by arrow 430. Plate 401 is secured to a pair of rotatable mounted arms 432, 434 which travel in a clockwise direction and which move plate 401 in a clockwise motion. Plate 402 is connected to a pair of rotary arms 436, 438, which travel in a counter clockwise direction moving plate 402 in a counter clockwise motion. Thus, as the starting substrate 406 moves between plates 401 and 402 in the direction indicated by the arrow 430, a portion of the starting substrate between the plates is formed and then released such that the plates 401 and 402 may come together and form another section of starting substrate 406. This method has the benefit of allowing virtually any pattern of any complexity to be formed in a continuous process e.g. uni-directional, bi-directional and multi-directional patterns. The energy is added to the process in a preferred embodiment by heating plate 401 and/or plate 402. Alternatively, the substrate can be heated prior to the texturing process.

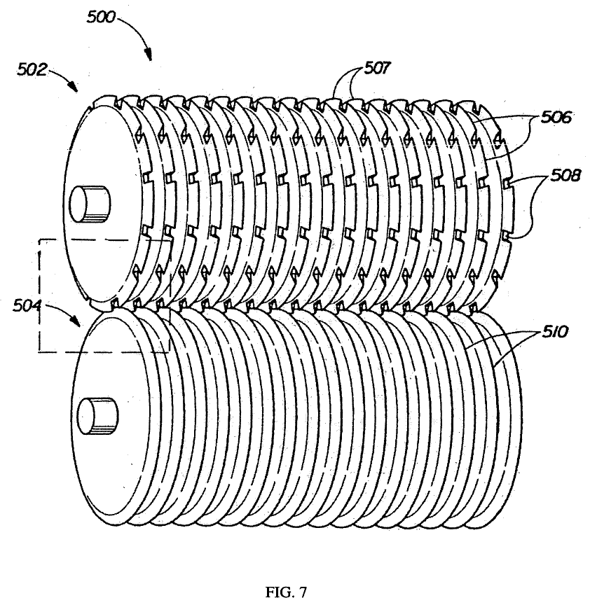

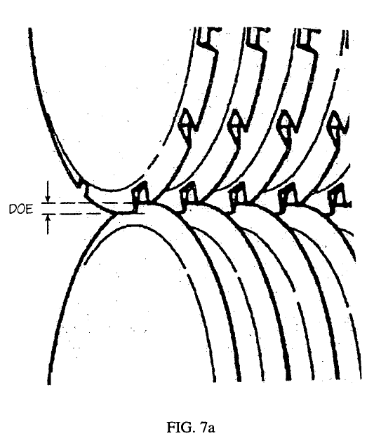

FIG. 7 shows another apparatus generally indicated as 500 for continuously forming the substrate of the present invention. Apparatus 500 includes a pair of rolls 502 and 504. Roll 502 includes a plurality of toothed regions 506 and a plurality of grooved regions 508 that extend substantially parallel to a longitudinal axis running through the center of the cylindrical roll 502. Toothed regions 506 include a plurality of teeth 507. Roll 504 includes a plurality of teeth 510, which mesh with teeth 507 on roll 502. As a starting substrate is passed between intermeshing rolls 502 and 504, the grooved regions 508 will leave portions of the starting substrate unformed producing the first regions of the substrate of the present invention. The portion of the starting substrate passing between toothed regions 506 and 510 will be formed by teeth 507 and 510, respectively, producing the second regions of the substrates of the present invention, and more specifically the protruding elements of the present invention. In a preferred embodiment, to lock fibers in the second regions of the textured substrate, the rolls 504 and 502 are heated to about the melting temperature of one of the components of the starting substrate. In another preferred embodiment, at least one of the rolls 504 and 502 is heated to about the melting temperature of one of the components of the starting substrate. Alternative, the starting substrate could be heated prior to the texturing process.

Alternatively, roll 504 may consist of soft rubber. As the starting substrate is passed between toothed roll 502 and rubber roll 504 the starting substrate is mechanically formed into the pattern provided by toothed roll 502. The substrate within the grooved regions 508 will remain unformed, while the starting substrate within the toothed regions 506 will be formed producing the second regions of the substrate of the present invention, and more specifically the protruding elements of the present invention.

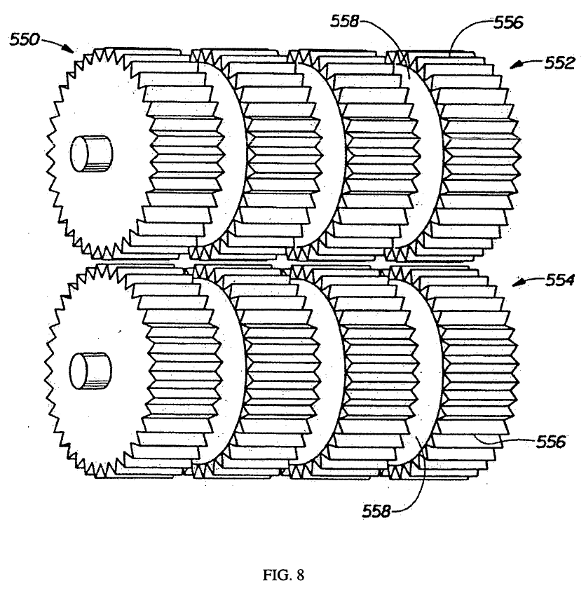

FIG. 8 shows an alternative apparatus generally indicated as 550 for forming the starting substrate into a textured substrate. Apparatus 550 includes a pair of rolls 552, 554. Rolls 552 and 554 each have a plurality of toothed regions 556 and grooved regions 558 extending about the circumference of rolls 552, 554 respectively. As the starting substrate passes between 552, 554 the grooved regions 558 will leave portions of the starting substrate unformed, while the portions of the starting substrate passing between toothed regions 556 will be formed producing the second regions of the substrates of the present invention, and more specifically the protruding elements of the present invention. In a preferred embodiment, to lock fibers in the second regions of the textured substrate, the rolls 552 and 554 are heated to about the melting temperature of one of the components of the starting substrate. In another preferred embodiment, at least one of the rolls 552 and 554 is heated to about the melting temperature of one of the components of the starting substrate.

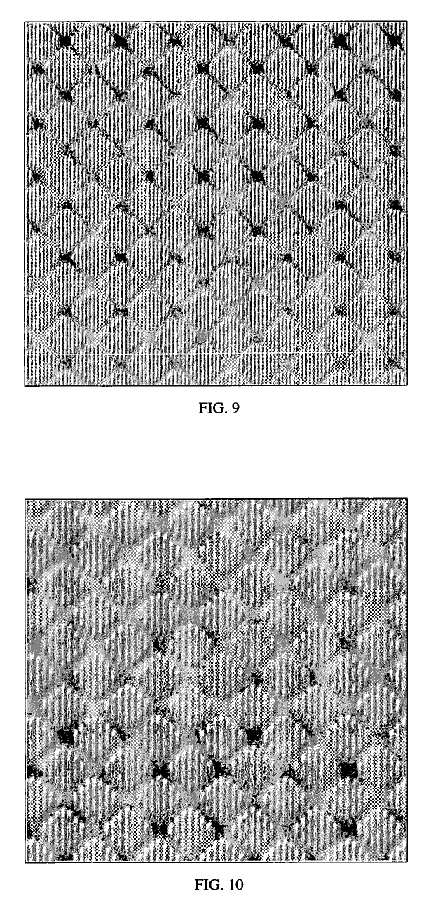

The height, frequency, and length of the protruding elements of the substrate is dependent on: (1) tooth pitch, meaning the distance from tooth tip to tooth tip; (2) depth of engagement (see distance DOE in FIG. 7a), meaning the extent to which the toothed and grooved regions of the two rolls overlap; (3) substrate properties (e.g., basis weight, caliper, number of fibers, fiber diameter, fiber types, etc.); and (4) length of teeth (see length L in FIG. 4). During the mechanical manipulation process, the starting substrate is traveling between the upper and lower rolls. While the starting substrate travels between the rolls described, the starting substrate becomes “anchored” between the tips of teeth on either roll (i.e., when the starting substrate cannot move in the direction perpendicular to movement of starting substrate through the rolls). From a hardware point of view, the point when starting substrate becomes “anchored” depends on (1) the tooth pitch and (2) depth of engagement. Typically, the smaller the tooth pitch and larger the depth of engagement, leads to quicker “anchoring” of the starting substrate between the tips of teeth on either roll and thus taller and more frequent protruding elements. Hence, in order to produce a substrate with protruding elements, but not being bound to a specific tooth pitch and starting substrate, the depth of engagement of the toothed and grooved regions is preferably in excess of 0.25 mm. By changing the length of the tooth (length L in FIG. 4) in a given pattern, different shapes of the second regions can be produced in the substrate—for example, diamond shapes of the second regions in FIGS. 9 and 10 are produced by linearly varying the tooth length, or a constant tooth length can produce the following texture patterns but are not limited to a striped, rectangular, wavy, or a square pattern depending on the dimensions and shape of the first regions. Types of pattern shapes include but are not limited diamond, square, rectangle, circle, ellipse, waves, trapezium, stripes, etc. The dimensions of pattern shape depend on the length of tooth selected. Without being bound by theory, it is believed that maximum size of the tooth length is dependent on the tooth pitch to maintain the 3-dimensionality of the protruding elements of the second regions. Preferably, the tooth length is between about 0.5 mm and about 15 times the tooth pitch, more preferably between about 1 mm and about 12 times the tooth pitch, and most preferably between about 2 mm and 10 times the tooth pitch. The tooth length can be designed based on the substrate surface textures, which are created by the size and shape of protruding elements, meeting the consumer needs of a wet wipe. It will be readily apparent to those skilled in the art that various tooth shapes, sizes, pitches, depth-of-engagements, and patterns can be designed to create a consumer-preferred substrate. These design modifications are expressly intended to be within the scope of the present invention.

It is clear from the above process that the first regions result from contact with the grooved regions of the roll and are thus unformed and substantially planar. However it may also be envisioned that the first regions comprise a comparatively minor level of formation. In this case, the grooves of the roll may be shallow or comprise an irregular surface such that when the starting substrate is fed through the machinery, the first regions comprise a corresponding irregular surface. Alternatively it may be envisioned that the starting substrate may be fed through a series of manipulation processes. In at least one of these processes, the first regions are manipulated so as to be minorly formed. Subjecting the starting substrate to a series of texturing processes allows the manufacturer to produce a substrate comprising more than one pattern. Thus, a first pattern is formed during a first texturing step and a second pattern is formed during a second texturizing step. It is also conceivable that more than two patterns are applied to the substrate. Other processing variation include embossing the substrate prior to the process for texturizing the first and second regions. Preferably, a substrate comprising a texture of first and second regions is subsequently embossed. This enables the embossed pattern to be on top of the texture pattern and easier to see.

In order to make the process feasible for mass production of commercial interest, the process would desirably run at a minimum speed of approximately 20 meters/minute. Suitable starting substrates for use in such high speed manipulation of the web(s) are those that can be manipulated at said minimum speed without tearing, perforating, creating holes and/or substantially unacceptable thin regions (i.e. less opaque, lower fiber concentration) in the substrate.

The processes described in the above paragraphs detail known texturing processes, with the exception of adding the energy. A wet wipe produced by any of previous texturing processes without the addition of energy will form a textured wipe but when the wipe is wet, the texture and thickness will be significantly reduced depending upon the fibers comprising the substrate of the wet wipe. The addition of the energy will enable a textured wipe that is wet to retain a significant amount of its texture and thickness, thereby enabling the formation a textured wet wipe.

Substrate Composition

The first and second regions are preferably comprised of the same material composition. The substrate of the present invention is made from at least one fibrous web. It is envisioned that the substrate according to the present invention may be a single fibrous web that has undergone the mechanical manipulation to form the first and second regions of the substrate. Alternatively, it can equally be envisioned that the substrate may be composed of a laminate of at least two, more preferably at least three or even more webs, wherein at least one web is a fibrous web. The laminate of webs may be compiled prior to being subjected to the mechanical manipulation to form the first and second regions of the substrate as above. Alternatively, the laminate of webs may be compiled at the point where the webs are fed into the machinery. Further still, it can be envisioned that the substrate composed of a single fibrous web or a laminate of two or more webs is subjected to the mechanical manipulation above, and is then used as a component of a more complex wet wipe structure.

The starting substrates of the present invention are formed by any of the following processes: carding, airlaid, spunbond, meltblown, coform, wetlaid, and mixtures thereof. The starting substrates of the present invention are consolidated by any of the following processes: hydroentanglement, thermal calender bonding, through air thermal bonding, chemical bonding, needlepunching, and mixtures thereof. As used herein, the term “hydroentanglement” means generally a process of treatment of a starting substrate wherein a layer of loose fibrous material is supported on an apertured member and is subjected to water pressures sufficiently great to cause the individual fibers to mechanically entangle with other fibers and possibly other web layers of a substrate. The apertured member can be made from a woven screen, a perforated metal plate, etc. The preferred method of making the nonwoven substrate of present invention is carding followed by hydroentanglement. The substrates of the present invention preferably have a dry basis weight of from 15 to 150 grams/meter², more preferably from 20 to 100 grams/meter² and most preferably from 30 to 90 grams/meter².

Fibers and materials suitable for making the starting substrates used in the production of the substrates of the present invention are selected from the group consisting of: thermoplastic fibers, natural fibers, cellulosic fibers, and mixtures thereof. Types of “thermoplastic fibers” include but are not limited to fibers made of polyolefins (e.g., polyethylene and polypropylene), polyesters, polyamides, polyimides, polyacrylates, polyacrylonitrile, polylactic acid, polyhydroxyalkanoate, polyvinyl alcohol, polystyrene, polyaramids, polysaccharides and blends and co-polymers thereof. Fibers may comprise single or multi-components of said thermoplastic polymers. Examples of multicomponent fibers include but are not limited to fibers comprising a sheath/core, side-by-side, islands-in-the-sea construction of at least two different materials selected from the thermoplastic fibers. Types of “cellulosic” fibers include but are not limited to wood pulp, rayon, lyocell, cellulose acetate, cellulose esters and mixtures thereof. Types of natural fibers include but are not limited to cotton, wool, silk, jute, linen, ramie, hemp, flax, camel hair, kenaf, and the like. Preferred fibers for making the substrates of the present invention are polyolefin fibers, cellulosic fibers, and mixtures thereof.

The fiber composition of the nonwoven substrate will depend upon the desired finished product use, basis weight, and form of energy used to reinforce the fibers in the second region, among other things. When heat is used as the reinforcing means, preferably the nonwoven substrate will comprise greater than about 20% thermoplastic fibers, more preferably greater than about 40% thermoplastic fibers, and most preferably greater than about 50% thermoplastic fibers. The nonwoven substrate may comprise 100% of thermoplastic fibers. The determination as to the composition of the nonwoven substrate will depend upon the use of the wipe and the desired characteristics such as softness, flushability, biodegradability, strength, abrasivity, and other desired properties.

The starting substrates having locks prior to forming the first and second regions of substrate of the present invention can comprise fibers with various different cross-sectional shapes and controlled surface frictional properties. Such starting substrate is formed by carding and consolidated by hydroentanglement. Without being bound by theory, it is believed that various different cross-sectional shapes and controlled surface frictional properties of fibers provide stronger frictional entanglement or frictional interlocking of fibers during hydroentangling consolidation process. Said stronger entanglement can be preserved during the forming process and may help to provide extra strength to the locking of fibers in the second regions.

Preferred starting substrates are composed of a single fibrous web made from a carded-hydroentangled web comprising of polypropylene and rayon fibers in at least two different relative compositions. A preferred starting substrate of the present invention is about 60 grams/meter² basis weight carded-hydroentangled Fibrella 3160 nonwoven substrate from J. W. Suominen, Finland, comprising homogenously distributed 60 weight % polypropylene fibers and 40 weight % viscose rayon fibers. Another preferred starting substrate is about 60 grams/meter² basis weight carded-hydroentangled Fibrella 3173 nonwoven substrate from J. W. Suominen, Finland, comprising 75 weight % polypropylene fibers and 25 weight % viscose rayon fibers. In this preferred substrate, three carded layers of polypropylene and viscose rayon fibers are stacked up and hydroentangled together. The top and bottom layers of this preferred substrate comprise homogenously distributed equal amounts of polypropylene fibers and viscose rayon fibers, while the middle layer comprises only polypropylene fibers.

In another preferred embodiment the starting substrate is about 70 grams/meter² basis weight of a laminate of two different fibrous webs stacked and consolidated together by hydroentangling. This preferred embodiment comprises three layers: top and bottom layers are carded layers (20 grams per square meter each) of homogenously blended 60% polypropylene and 40% viscose rayon fibers by weight; the middle layer is 30 grams per square meter spunbond comprising 50/50 sheath/core polyethylene/polypropylene bicomponent fibers. All three layers are stacked up and hydroentangled together. After the mechanical manipulation, as described above, a substrate of the present invention is formed, wherein the middle spunbond layer of the starting substrate with thermal bonds provides the fiber locking necessary to keep the second regions distinct from the first regions.

In addition to fibers, the starting substrates of the present invention may contain additives that can be activated by the addition of energy (as mentioned above) during the process of creating the first and second regions of the substrate of the present invention. Types of additives include but are not limited to binders, adhesives, chemicals, monomers, melt additives, and surface finishes on the fibers of the starting substrate. Types of activation of additives include but are not limited to melting, cross-linking, polymerization, chemical bonding, and ionic bonding. Without being bound by theory, it is believed that these additives on activation, during the texturing process, provide the locking of fibers in the second regions of the substrate of the present invention. It will be readily apparent to those skilled in the art that the starting substrates can comprise components that can be easily activated during the texturing process, as described above, to create the substrates of the present invention. These components of the starting substrates are expressly intended to be within the scope of the present invention.

Liquid and Lotion

A lotion, which is preferably a liquid, is added to the nonwoven substrate. A liquid can be any desired fluid, such as water or a lotion. The amount of lotion added to the substrate is in the range of from about 10% to about 500% by weight of the dry nonwoven substrate. Typically, a substrate is considered wet when comprising greater than about 20% of a liquid. Many uses of the wipes desire more than 65% of a liquid. The amount of lotion will depend upon the intended use of the wipe and if the manufacturer or consumer is adding the liquid. The lotion can be added as a hotmelt liquid paste so that it solidifies upon cooling, or can be added as a liquid followed by drying to a lower water content.

The lotion can be an aqueous lotion, and may include skin-conditioning ingredients. One preferred lotion comprises polymeric emulsifiers, such as sodium acrylates, and silicon oil, such as dimethicone in an oil-in-water emulsion type formulation. Lotions can also include one or more surface-active materials (surfactants) which can enhance cleansing and/or promote generation of a lather. The lotion can also include preservative and fragrance ingredients.

In one preferred formulation, the lotion is preferably at least about 85 percent by weight water, more preferably at least about 90 percent by weight water, and still more preferably at least about 95 by weight water. If a consumer is adding the liquid, the lotion ingredients can be added to the substrate is a dry form and then a consumer adds the liquid, typically water, to form the lotion. A currently preferred lotion is an oil-in-water emulsion type formulation comprising a polymeric emulsifier, preferably sodium acrylates, and silicon oil, preferably dimethicone. The lotion can comprise an aqueous solution comprising a surfactant selected from the group consisting of phosphate quaternary amine compounds and non-ionic surfactants, and effective amounts of a second ingredient selected from the group consisting of non-cellulosic organic water soluble polymers and alkoxylated alcohols. The amount of these components can be adjusted in effective amounts to provide varying levels of adhesional wetting to account for various fold patterns and dispensing openings to deliver reliable wet wipe dispensing. In another embodiment, the lotion can comprise a non-ionic surfactant that is a block copolymer of propylene oxide and ethylene oxide. The propylene oxide block is sandwiched between two ethylene oxide blocks selected from the group consisting of Poloxamer 101-Poloxamer 407. A suitable nonionic surfactant is commercially available as Pluronic 62 brand available from BASF Corporation, Mount Olive, N.J. The lotion preferably comprises less than about 3 percent by weight of the nonionic surfactant. More preferably, the lotion can comprise less than about 1 percent by weight of the nonionic surfactant. Even more preferably, the lotion comprises between about 0.2 and about 0.3 percent by weight of the nonionic surfactant. In another preferred embodiment, the lotion comprises an inner salt of fatty quaternary amines as a surfactant and a sulfonate of a fatty quaternary as a cosurfactant. The surfactant can be selected from the group consisting of Caprylamidopropyl Betaines, Cocoamidopropyl Betaines, Lauramidopropyl Betaine, Oleamidopropyl Bataine, or Isosteramidopropyl Betaine commercially available as Mackam: OAB, 35, L, J, DZ, LMB, and ISA from McIntyre Group Ltd., Governors Highway, University Park, Ill. A suitable cosurfactant is Cocamidopropyl Hydroxysultaine commercially available as MackamCBS-50G from McIntyre Group Ltd., Governors Highway, University Park, Ill. The lotion preferably comprises less than about 3 percent by weight of the inner salt of fatty quaternary amines and less than about 1 percent by weight of the sulfonate of a fatty quaternary. More preferably, the lotion can comprise less than about 1 percent by weight of the inner salt of fatty quaternary amines compound and less than about 0.7 by weight of the sulfonate of a fatty quaternary. Still more preferably, the lotion comprises between about 0.15 and about 0.36 percent by weight of the inner salt of fatty quaternary amines compound and between about 0.1 and about 0.36 percent by weight of the sulfonate of a fatty quaternary. The lotion preferably also comprises one or more of the following: an effective amount of a preservative, an effective amount of a humectant, an effective amount of an emollient; an effective amount of a fragrance, and an effective amount of a fragrance solubilizer. As used herein, an emollient is a material that softens, soothes, supples, coats, lubricates, or moisturizes the skin. The term emollient includes, but is not limited to, conventional lipid materials (e.g. fats, waxes), polar lipids (lipids that have been hydrophilically modified to render them more water soluble), silicones, hydrocarbons, and other solvent materials. Emollients useful in the present invention can be petroleum based, fatty acid ester type, alkyl ethoxylate type, fatty acid ester ethoxylates, fatty alcohol type, polysiloxane type, mucopolysaccharides, or mixtures thereof. Humectants are hygroscopic materials that function to draw water into the stratum comeum to hydrate the skin. The water may come from the dermis or from the atmosphere. Examples of humectants include glycerin, propylene glycol, and phospholipids. Fragrance components, such as perfumes, include, but are not limited to water insoluble oils, including essential oils. Fragrance solubilizer are components which reduce the tendency of the water insoluble fragrance component to precipitate from the lotion. Examples of fragrance solubilizer include alcohols such as ethanol, isopropanol, benzyl alcohol, and phenoxyethanol; any high HLB (HLB greater than 13) emulsifier, including but not limited to polysorbate; and highly ethoxylated acids and alcohols. Preservatives prevent the growth of microorganisms in the liquid lotion and/or the substrate. Generally, such preservatives are hydrophobic or hydrophilic organic molecules. Suitable preservatives include, but are not limited to parabens, such as methyl parabens, propyl parabens, and combinations thereof. The lotion can also comprise an effective amount of a kerotolytic for providing the function of encouraging healing of the skin. An especially preferred kerotolytic is Allantoin ((2,5-Dioxo-4-Imidazolidinyl)Urea), a heterocyclic organic compound having an empirical formula C4H6 N4 O3. Allantoin is commercially available from Tri-K Industries of Emerson, N.J. It is well recognized that the long term wear of disposable absorbent structures, such as disposable diapers, may lead to skin which is compromised in terms of being over hydrated. It is generally known that hyper hydrated skin is more susceptible to skin disorders, including heat rash, abrasion, pressure marks and skin barrier loss. For example, 21 CFR 333.503 defines diaper rash as an inflammatory skin condition in the diaper area (perineum, buttocks, lower abdomen, and inner thighs) caused by one or more of the following factors: moisture, occlusion, chafing, continued contact with urine or feces, or mechanical or chemical irritation. A pre-moistened wipe according to the present invention can include an effective amount of allantoin for encouraging the healing of skin, such as skin that is over hydrated. U.S. Pat. No. 5,534,265 issued Jul. 9, 1996; U.S. Pat. No. 5,043,155 issued Aug. 27, 1991; and U.S. Pat. No. 5,648,083 issued Jul. 15, 1997 are incorporated herein by reference for the purpose of disclosing additional lotion ingredients. The lotion can further comprise between about 0.1 and about 3 percent by weight Allantoin, and about 0.1 to about 10 percent by weight of an aloe extract, such as aloe vera, which can serve as an emollient. Aloe vera extract is available in the form of a concentrated powder from the Rita Corporation of Woodstock, Ill.

Not all wet wipe lotions are designed specifically for hygiene applications. Some wet wipes are intended for cleaning non-human surfaces. Such surfaces would include, but are not limited to, floors, countertops, cabinets, appliances, woodwork, sinks, tubs, dishes, showers, tile, glass, and mirrors.

An example of a lotion that is suitable for non-human surface wipes is a mixture of approximately 90.5% water with the following ingredients added: C10 Amine Oxide, Neodol 91.5, Popylen-Glycol Butyl Ether, Ethanol, 2-ethyl-hexyl sulphate, Silicon AF, and a fragrance.

Applications

The present invention is suitable for a wide array of wet wipe applications. For example, three currently marketed wet wipe products are baby wipes, surface cleaning wipes, and facial cleansing wipes.

Baby wipes are often used to clean an infant's skin during a diaper change. Consumers expect baby wipes to provide gentle cleaning of the baby. The present invention accomplishes this. The additional wet thickness observed in the second region, while not increasing the dry basis weight results in a decrease in density in the wipe. With this decrease in density locked in place, the wet wipe has increased resiliency, a key measure of gentleness. Since the structure in the second region is non-planar, there are three-dimensional volumes where soil to be cleaned can stored. This results in improved cleaning. Hence, gentler and more thorough cleaning can be provided.

Surface cleaning wipes work by various means, including but not limited to mechanical abrasive action to loosen soil from a surface, solublization of soil from the lotion in the wet wipe, and collection and entrapment of soil into the structure of the wet wipe. Since the structure in the second region is non-planar, there are three-dimensional volumes where soil can be collected and entrapped. Additionally, if one of the preferred embodiments is employed and thermal energy is used to lock fibers in the second region, and if a the nonwoven substrate is subjected to temperatures substantially near to the melting point of at least one of the thermoplastic fibers, then a relatively highly abrasive surface can be produced. This relatively high friction surface can improve cleaning from surfaces.

Facial cleansing wipes can be produced as pre-moistened wet wipes or packaged as dry wipes where the consumer adds a liquid such as lotion or water. One desirable attribute of facial cleansing wipes is that they provide a relatively abrasive surface to help exfoliate skin cells and also provide a relatively soft surface for gentle cleansing. The present invention can accomplish these two tasks concurrently. It is possible to create the protruding elements of the second region on only one side of the nonwoven substrate and not have protruding elements of the second region on the other side of the nonwoven substrate. If the protruding elements of the second region are created by a preferred embodiment where thermal energy is used to lock fibers in the second region, and if a the nonwoven substrate is subjected to temperatures substantially near to the melting point of at least one of the thermoplastic fibers, then a relatively high abrasion surface can be produced on one side of the nonwoven substrate and leaving a relatively soft, non-abrasive surface on the opposite side.

### Test Methods

Basis Weight: Basis weight is defined as mass per unit area of a substrate.

PRINCIPLE: Measurement of the area and mass of a specimen substrate and calculation of its mass per unit area in grams per square meter.

Apparatus:

1. Apparatus for cutting the test pieces, chosen from the following.

a. Die, which cuts a test piece of an area of at least 0.036 meter² (180 mm×200 mm).

b. Template, with an area of at least 0.036 meter² (180 mm×200 mm) and a razor blade.

c. Steel rule, accurately graduated in millimeters, and a razor blade.

2. A balance, capable of determining the mass of a test piece to an accuracy of +/−0.1% of the determined mass.

PREPARATION OF TEST PIECES: From each specimen sample, cut at least five test pieces, each of at least 0.036 meter² (180 mm×200 mm) using either the die, or the template and a sharp razor blade, making sure that the test piece does not stretch.

PROCEDURE: Determine the mass of each of the test piece using a balance.

RESULTS: Basis weight is calculated by dividing the measured mass in grams of the substrate with the cut area (0.036 meter²) as grams/meter². The average basis weight of the specimen substrate is calculated from five replicate test pieces. As described in the present invention, basis weight of the starting substrate and the textured substrate is measured in their dry states prior to wetting with a liquid.

Wet Thickness: Wet thickness is distance between face and the back of a wet nonwoven substrate. EDANA Test Method 30.5-99 is used to measure the wet thickness of a wet nonwoven substrate of the present invention.

PRINCIPLE: Measurement of the thickness of a wet nonwoven substrate as the distance between the reference plate on which the nonwoven rests and a parallel presser-foot that exerts a specified pressure on the area under test. The wet thickness can be measured on a starting substrate or textured substrate.

APPARATUS: Two circular horizontal plates, attached to a stand, comprising an upper plate, or presser-foot, capable of moving vertically and having an area of approximately 2,500 mm², and a reference plate having a plane surface of diameter at least 50 mm greater than that of the presser-foot. A measuring device, having a scale with 0.01 mm graduations, for measuring the distance between the reference plate and the presser-foot is used. Thwing-Albert ProGage Thickness tester, calibrated to EDANA Test Method 30.5-99, meets the measuring apparatus requirements. This instrument is used to measure wet thickness of the substrate of present invention.

PREPARATION OF TEST PIECES: From each wet specimen sample, cut at least five test pieces, each of at least 2,500 mm using either the die, or the template and a sharp razor blade, making sure that the test piece does not stretch.

PROCEDURE: Using the apparatus specified above, adjust the load on the presser-foot according to the manufacturer's instructions to give a uniform pressure of 0.5 kPa and set the measuring device to zero position. Calibrate thickness every test day with a 0.4 inch steel gage block. Raise the presser-foot, and position the test piece centrally with respect to the presser-foot, and without tension, on the reference plate. Lower the presser-foot carefully until contact is made with the test piece, and leave in contact for 10 seconds. Note the reading, in millimeters and raise the presser-foot to remove the test piece. Repeat the procedure on other 4 test pieces.

RESULTS: Calculate mean thickness of the specimen in mm. For the preferred wet substrate embodiment, a test piece, about 10,000 mm², is cut and its wet thickness is measured at three different positions in diagonal direction—one at each opposite corner and a third in the center of the test piece. A total of five replicate test pieces are used for calculating mean thickness.

Wet Structural Permanence: Wet structural permanence is defined as the ratio of wet thickness after the removal of external forces deforming a textured wet substrate to the wet thickness after the removal of external forces deforming a starting (non-textured) substrate.

PRINCIPLE: Measurement of wet thickness of starting and textured substrates. The measurements are taken on both substrate before and after subjecting to compression for given period of time.

Apparatus:

1. Two 3″×5″ Plexiglas® plates, each weighing about 0.5 lb

2. 2.6±0.01 lb compression weight, each 3″×5″ in area (representing conditions a wet wipe may experience in packaging and shipping, equivalent to about 0.2 psi (about 1.4 kPa) compression pressure)

3. Ziploc® bag—big enough to fit wipe stack with Plexiglas® plates.

4. Thwing-Albert ProGage Thickness tester—using EDANA test method 30.5-99 (as described in Wet Thickness test method.)

PREPARATION OF TEST PIECES: For each wet substrate specimen (starting substrate and textured substrates), cut 13 samples each 3″×5″ in area using either a die, or a template and a sharp razor blade, making sure that the test pieces do not stretch.

Procedure:

1. Take 5 test pieces from each specimen and label them 1 to 5. Keep rest 8 test pieces aside for the time being.

2. Measure and record the “initial” wet thickness of each of 5 labeled test piece using the Wet Thickness measurement method.

3. After measurement, neatly stack the 5 labeled test pieces along with the other 8 unlabeled test pieces with 4 unlabeled pieces are on the top of 5 labeled pieces and 4 unlabeled pieces on the bottom of 5 labeled pieces.

4. Place the stacked test pieces between two Plexiglas® plates with edges of the stack matching the edges of the plates. It may be easier to label the plates—top and bottom—to keep the stack in the same order.

5. Place complete test stack inside a Ziploc® bag and close tightly after carefully removing excess air from bag without putting any pressure on the sample stack.

6. Place 2.6 lb weight on the top of bagged test stack and keep the whole stack at room temperature for 5 days.

7. After 5 days, remove the test weight and carefully take out the test pieces from the Ziploc® bag. Measure and record the “final” wet thickness of each of the labeled test pieces using the Wet Thickness measurement method.

8. Repeat above steps for each wet specimen substrates. Use 4 replicate specimens for each substrate.

CALCULATIONS AND RESULTS: Calculate the initial and final average wet thickness of test pieces of each wet specimen substrate. Divide the average final wet thickness of the textured specimen substrate by that of the starting (non-textured) specimen substrate to evaluate the Wet Structural Permanence (after being subjected to hydrodynamic and compression forces) of the textured substrate of the present invention.

### EXAMPLES

### Examples 1-4

The following examples are non-limiting examples of nonwoven substrates of the present invention. Each initial nonwoven substrate is subjected to the method of texturizing in the static mode, as described in the detailed description of the invention (see FIG. 5), to form the first region and the reinforced second region. An Airam Model ATP-1585 pneumatic press is used to make these examples. A wipe with dimensions of 180 mm by 200 mm is placed between plates 401 and 402 and the plates are then brought together under a pressure indicated as 80 psi on the pneumatic press. The nonwoven substrates are then made into wet wipes by uniformly applying approximately 3.15 grams of lotion per gram of dry substrate. The lotion used in these examples is a mixture of approximately 95% water with the following ingredients added: Polysorbate 20, Acrylates/Vinyl Isodecanoate Crosspolymer, Disodium EDTA, Dimethicone, Methylparaben, Propylparaben, Ethylparaben, Pehenoxyethanol, Propylene Glycol, Sodium Hydroxide, and Fragrance. Non-limiting applications of nonwoven substrates described in Examples 1-4 include baby wipes, facial cleansing wipes, surface cleaning wipes, polishing wipes, and personal hygiene wipes.

### Example 1

An initial nonwoven, Fibrella 3173 from J.W. Suominen Oy, Nakkila, Finland, is used. Fibrella 3173 is a 60 gsm carded nonwoven substrate made from a fibrous blend of approximately 73% polypropylene and approximately 27% viscose rayon. The polypropylene has a denier of 1.5 dpf and a length of 40 mm. This viscose rayon has a denier of 1.5 dpf and a length of 40 mm. During the carding process, three discrete layers of carded material are layered one on top of each another. Each of the three layers is approximately equal in basis weight. Each of the two outer layers has a blend of approximately 60% polypropylene and 40% viscose rayon. The center layer is made of 100% polypropylene. This carded material is then hydroentangled, and dried to form the initial nonwoven.

For comparison, two substrates made as described above were tested. The Control substrate was processed according to a standard texturizing method. The Reinforced substrate was processed according to the same texturizing method as the Control substrate but heat was added to provide the reinforced second region.

| | Control | Reinforced |
|---|---|---|
| Condition | | |
| Temperature [° C.] | 25 | 160 |
| Dwell Time [sec] | 0.1 | 0.1 |
| Pattern Pitch [mm] | 2.5 | 2.5 |
| Depth of Engagement [mm] | 1.8 | 1.8 |
| Wet Thickness (mm) | | |
| Base (non-textured) substrate | 0.49 | 0.49 |
| Textured substrate | 0.56 | 0.89 |
| Base substrate (non-textured) after | 0.45 | 0.45 |
| compression | | |
| Textured substrate after compression | 0.49 | 0.61 |

Results of wet thickness (prior to compression): The wet thickness of the Reinforced (textured) substrate is about 0.89 mm. This represents about an 82% increase in wet thickness compared to the base (non-textured) substrate and about 59% increase in wet thickness compared to the Control (textured) substrate.

Results of wet structural performance (wet thickness after compression): The wet thickness of the Reinforced (textured) substrate is 0.61 mm. This represents about a 36% increase in wet thickness compared to the base (non-textured) substrate and about a 24% increase in wet thickness compared to the Control (textured) substrate. The wet structural permanence of the Reinforced (textured) substrate is 1.36, while the wet structural permanence of the Control (textured) substrate is 1.09.

### Example 2

An initial nonwoven, Softex® from BBA Nonwovens, Simpsonville, S.C., USA, is used. This grade of Softex® is a 60 gsm spunbond nonwoven. The filaments are biconstituent, with a polyethylene sheath and a polypropylene core. The weight percentage of the polyethylene sheath is approximately 50% of the entire filament. The base nonwoven is then wet. The wet thickness of this base (non-textured) nonwoven is about 0.49 mm.

The process conditions used to create a first region and a reinforced second region are:

| | |
|---|---|
| Temperature [° C.] | 80 |
| Dwell Time [sec] | 0.4 |
| Pattern Pitch [mm] | 2.5 |
| Depth of Engagement [mm] | 1.8 |

The wet thickness of the reinforced textured nonwoven is about 1.36 mm, which represents about 178% increase in wet thickness compared to the base (non-textured) nonwoven.

### Example 3

An initial nonwoven is 64 gsm and is made from a fibrous blend of approximately 86% Southern softwood kraft fluff pulp and 14% polyester staple fiber. These fibers are air laid to form a mat and then approximately 14% add-on of a styrene butadiene resin is applied to the web by hydraulic nozzles. The nonwoven substrate is then dried to form the initial nonwoven. For comparison, two substrates produced as described above were tested. The Control substrate was processed according to a standard texturizing method. The Reinforced substrate was processed according to the same texturizing method as the Control substrate but heat was added to provide the reinforced second region.

| | Control | Reinforced |
|---|---|---|
| Condition | | |
| Temperature [° C.] | 25 | 160 |
| Dwell Time [sec] | 0.4 | 0.4 |
| Pattern Pitch [mm] | 2.5 | 2.5 |
| Depth of Engagement [mm] | 1.4 | 1.4 |
| Wet Thickness (mm) | | |
| Base (non-textured) substrate | 0.65 | 0.65 |
| Textured substrate | 0.73 | 0.86 |
| Base substrate (non-textured) after | 0.61 | 0.61 |
| compression | | |
| Textured substrate after compression | 0.55 | 0.66 |

Results of wet thickness (prior to compression): The wet thickness of the Reinforced (textured) substrate is about 0.86 mm. This represents about a 56% increase in wet thickness compared to the base (non-textured) substrate and about an 18% increase in wet thickness compared to the Control (textured) substrate.

Results of wet structural performance (wet thickness after compression): The wet thickness of the Reinforced (textured) substrate is 0.66 mm. This represents about an 8% increase in wet thickness compared to the base (non-textured) substrate and about a 20% increase in wet thickness compared to the Control (textured) substrate. The wet structural permanence of the Reinforced (textured) substrate is 1.10, while the wet structural permanence of the Control (textured) substrate is 0.90.

### Example 4

An initial nonwoven is 60 gsm and is made from a fibrous blend of approximately 30% polypropylene, approximately 40% viscose rayon, and approximately 30% polypropylene/polyethylene biconstituent. The polypropylene has a denier of 1.5 dpf and a length of 40 mm. This viscose rayon has a denier of 1.5 dpf and a length of 40 mm. The biconstituent fiber has a polyethylene sheath and a polypropylene core, each constituent approximately 50% by weight of the fiber. The biconstituent fiber has a denier of 1.5 dpf and a length of 40 mm. These fibers are uniformly blended, carded, hydroentangled, and dried to form a nonwoven.

The initial nonwoven is then wet. The wet thickness of this initial nonwoven is about 0.47 mm.

The process conditions used to create a first region and a reinforced second region are:

| | |
|---|---|
| Temperature [° C.] | 125 |
| Dwell Time [sec] | 0.3 |
| Pattern Pitch [mm] | 3.0 |
| Depth of Engagement [mm] | 1.4 |

The wet thickness of the reinforced textured nonwoven is about 0.85 mm, which represents about 81% increase in wet thickness, compared to the initial nonwoven.

### Example 5

A starting nonwoven, Fibrella 3173, from J.W. Suominen Oy, Nakkila, Finland, is used. Fibrella 3173 is a 60 gsm carded nonwoven substrate made from a fibrous blend of approximately 73% polypropylene and approximately 27% viscose rayon. The polypropylene has a denier of 1.5 dpf and a length of 40 mm. This viscose rayon has a denier of 1.5 dpf and a length of 40 mm. During the carding process, three discrete layers of carded material are layered one on top of each another. Each of the three layers is approximately equal in basis weight. Each of the two outer layers has a blend of approximately 60% polypropylene and 40% viscose rayon. The center layer is made of 100% polypropylene. This carded material is then hydroentangled, and dried to form the base nonwoven substrate.

The base nonwoven substrate is subjected to the method of texturizing in the static mode, as described in the detailed description of the invention (see FIG. 5), to form the first region and the reinforced second region. An Airam Model ATP-1585 pneumatic press is used to make these examples. A wipe with dimensions of 180 mm by 200 mm is placed between plates 401 and 402 and the plates are then brought together under a pressure indicated as 80 psi on the pneumatic press. The nonwoven substrates are then made into wet wipes by fully saturating the substrate by applying approximately 5 grams of water per gram of dry substrate. The fully saturated substrate was then blotted to approximately 3.15 grams of water per gram of dry substrate. This method is used to simulate the use by a consumer adding water to a dry wipe to produce a wet wipe for use.

The process conditions used to create a first region and a reinforced second region are:

| | |
|---|---|
| Temperature [° C.] | 160 |
| Dwell Time [sec] | 1.0 |
| Pattern Pitch [mm] | 2.5 |
| Depth of Engagement [mm] | 1.8 |

The wet thickness of the base (non-textured) nonwoven is about 0.51 mm. The wet thickness of the reinforced textured nonwoven is about 1.67 mm, which represents about 227% increase in wet thickness compared to the base nonwoven.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is intended to cover in the appended claims all such changes and modifications that are within the scope of the invention.

What is claimed is:

1. A nonwoven substrate comprising at least one first region and at least one second region, wherein said second region comprises protruding elements locked by a reinforcing means selected from the group consisting of thermal bonding, chemical bonding, ionic bonding, adhesive bonding and combinations thereof.

2. The nonwoven substrate of claim 1 wherein the nonwoven substrate comprises at least about 20% thermoplastic material and the protruding elements of the second region are locked by means of thermal bonding.

3. The nonwoven substrate of claim 2 wherein the second region contains furrows and ridges.

4. The nonwoven substrate of claim 3 wherein the second region provides abrasivity when the nonwoven substrate is utilized in cleaning.

5. A wet wipe comprising the nonwoven substrate of claim 1.

6. A wet wipe of claim 5 wherein a liquid is applied to the nonwoven substrate before use.

7. A pre-moistened wet wipe of claim 5 wherein a liquid is applied to the nonwoven substrate prior to packaging.

8. The wet wipe of claim 5 wherein the nonwoven substrate is a laminate of webs comprising of at least one fibrous web.

9. The wet wipe of claim 7 wherein the amount of liquid added is in the range of from about 10% to about 500% by weight of the dry nonwoven substrate.

10. A wet wipe having a basis weight and a thickness, comprising:
a nonwoven substrate which is subject to a texturing process which does not increase the basis weight and
a liquid,
wherein the thickness of said wet wipe is at least about 30% greater than the thickness of the wet nonwoven substrate prior to being textured.

11. The wet wipe of claim 10 wherein the thickness of said wet wipe after being subject to external forces is at least about 30% greater than the thickness of the wet non-textured nonwoven substrate after being subject to external forces.

12. A process for forming a textured substrate comprising:
a) providing a nonwoven substrate,
b) feeding said nonwoven substrate through a pair of corresponding rolls wherein at least one roll comprises a plurality of toothed and grooved regions about the circumference of the roll and the roll is heated,
c) forming a first region of the nonwoven substrate from said grooved regions, and
d) forming a second region of the nonwoven substrate from said toothed region.

13. The process according to claim 12 wherein a liquid is applied to said textured substrate prior to packaging to form a premoistened wet wipe.

14. The process according to claim 12 wherein a liquid is applied to said textured substrate prior to use to form a wet wipe.

15. A process for forming a textured substrate comprising:
a. providing a nonwoven substrate,
b. feeding said nonwoven substrate through a pair of corresponding plates wherein at least one plate comprises a plurality of toothed and grooved regions about the surface of the plate and the plate is heated,
c. forming a first region of the nonwoven substrate from said grooved regions, and
d. forming a second region of the nonwoven substrate from said toothed region.

16. The process according to claim 15 wherein a liquid is applied to said textured substrate prior to packaging to form a premoistened wet wipe.

17. The process according to claim 15 wherein a liquid is applied to said textured substrate prior to use to form a wet wipe.

18. A process for forming a textured substrate comprising:
a. providing a heated nonwoven substrate,
b. feeding said heated nonwoven substrate through a pair of corresponding plates or rolls wherein at least one plate or roll comprises a plurality of toothed and grooved regions about the surface of the plate or roll,
c. forming a first region of the nonwoven substrate from said grooved regions, and
d. forming a second region of the nonwoven substrate from said toothed region.

19. A wet wipe produced according to the process of claim 12.

20. A wet wipe produced according to the process of claim 18.
